(19) United States
(12) Patent Application Publication
(10) Pub. No.: US 2013/0053307 A1
(43) Pub. Date: Feb. 28, 2013

(54) METHOD FOR WEIGHT LOSS AIDED BY ADMINISTRATION OF HUMAN CHORIONIC GONADOTROPIN

(71) Applicant: Sherri L. Emma, Toms River, NJ (US)

(72) Inventor: Sherri L. Emma, Toms River, NJ (US)

(21) Appl. No.: 13/593,954

(22) Filed: Aug. 24, 2012

Related U.S. Application Data
(60) Provisional application No. 61/526,731, filed on Aug. 24, 2011.

(51) Int. Cl. A61K 38/22; A61P 3/04

(52) U.S. Cl. 514/5.3

(57) ABSTRACT

Methods are provided for increased rates of weight loss while reducing the discomfort caused by dieting. An initial daily dose of HCG of 200 IU or greater is administered to a patient on a reduced calorie diet plan for weight loss. The reduced calorie diet plan includes limits on the amounts of protein, vegetables and fruits that may be eaten based upon caloric content and sugar levels in the vegetables and fruit. In one embodiment, the reduced calorie diet plan limits caloric intake to 500-800 calories per day. The patient is monitored and the daily HCG dose may be adjusted upward as needed to control hunger and maintain patient comfort.

### CROSS-REFERENCE TO RELATED APPLICATIONS

This application claims priority under 35 U.S.C. §119(e) to U.S. Provisional Application No. 61/526,731, filed on Aug. 24, 2011, the entire contents of which are hereby incorporated by reference.

### FIELD OF THE INVENTION

The present invention is generally directed to methods for losing weight aided by administration of human chorionic gonadotropin (HCG). The method involves the administration to a patient of a sufficient dose of HCG in combination with a carefully controlled diet plan to achieve a desired rate of weight loss. The method can also result in weight loss from certain areas of the body that improve overall appearance.

### BACKGROUND

In 1954, HCG was first documented as a weight loss aid by Dr. A. T. W. Simeons. Dr. Simeons conducted weight loss experiments with obese patients in an Italian clinic. From his findings he wrote the “Pounds and Inches” manuscript which outlined, in a combination of science, laymen's terms, and theory, his reasoning and findings of his treatments. In order to try to understand the science behind the method, roughly 22 studies were done in the 1970's and 1980's analyzing the use of HCG in weight loss. Of these studies, 8 were controlled and 14 were not. Meta-analysis of these studies was conducted in 1995 and found the majority of the studies to be either poorly powered or as producing unconvincing results. Some of the weaknesses of these studies were: (1) small study size, (2) low HCG dosing (125 IU), and (3) analysis of end point of difference in weight loss between HCG and placebo.

It is not surprising that use of HCG may not produce weight loss in a person that is different than that obtained using a placebo. HCG is not used to create or produce weight loss greater than diet alone. The weight loss produced during the HCG diet is brought about by caloric restriction. HCG itself is not a stimulant and therefore does not create a greater weight loss than caloric restriction alone. To the same point, patients that are not compliant will lose little, if any, weight. They must create a deficit between calories required and calories consumed to produce weight loss. This likely accounts for the studies failing to show any difference between HCG and placebo for weight loss.

On ordinary diets, people often cannot tolerate going below 1200 calories per day. For many people this does not produce an extreme calorie deficit. Therefore weight loss is produced at a very slow rate of a pound or two per week. Over time patients lose enthusiasm to continue and the slightest slip-up puts them right back where they were weeks before. The process results in a low probability of achieving significant weight loss.

One of the reasons most ordinary diets are not very low calorie diets (below 800 calories) is because hunger becomes unbearable. For many patients, this does not happen with the HCG treatments. The mechanism is yet to be discovered, but a Lancet Journal article in 1999 discussed the connection between high levels of HCG and hyperemesis gravidarium, thereby suggesting a mechanism involving the central emesis center of the brain. It is theoretically feasible that if this hormone can trigger nausea, then it can keep appetite away in lower doses.

Patients are comfortable in a number of ways on this protocol. Not only is hunger reduced, which is an important part of comfort, but there are also very few complaints of irritability or fatigue.

One of the most important effects of HCG in weight loss is muscle mass retention. There can be up to approximately a four-fold difference in ending muscle mass between patients that have undergone weight loss with HCG vs. without HCG. This may be because HCG, through elevation of testosterone, creates an anabolic hormonal state to counteract the catabolism of starvation. By counteracting catabolism that occurs during starvation, muscle mass and body tissues are spared from breakdown. Therefore the body turns to fat stores alone for energy use. This may explain the “selective fat loss” and “body-sculpting” Simeons observed but did not have scientific explanation for. When the body spares its lean tissues and selectively burns fat, this a much healthier way to lose weight because it does not pose the cardiac risk since the heart muscle is protected. Additionally, the electrolyte disturbances that often occur in the catabolic state of starvation do not occur.

In addition, because muscle is the powerhouse of a body's metabolism (fat is metabolically inactive), then muscle-sparing during dieting should put someone in a more favorable metabolic state post-diet than if they dieted without HCG and lost muscle mass. Some patients safely double their food consumption in the first week of maintenance with no regaining of weight.

In the prior dieting regimens using HCG, patients were given an initial HCG dose of 125 IU. This dose was increased in some patients, but the maximum dose of HCG used was 200 IU. This limit was due in part to concerns about potential side effects of HCG. Reports of HCG use for fertility (high doses—usually 5,000-10,000 IU) are accompanied by side effects such as water-retention, breast tenderness, blood clots, headaches, irritability, mood alterations. While prior diet plans using HCG provided benefits during weight loss in some patients, many continued to experience discomfort and difficulty maintaining the diet regimen. These patients may not achieve the desired weight loss due to their discomfort. Accordingly, it would be desirable to have a weight loss regimen using HCG that reduced patient discomfort and produced better weight loss results.

### SUMMARY OF THE INVENTION

The present invention is generally directed to a method for weight loss including administering an initial daily dose of HCG of 200 IU or greater to a patient, and providing a reduced calorie diet plan for the patient. The reduced calorie diet plan includes limits on the amounts of protein, vegetables and fruits that may be eaten based upon caloric content and sugar levels in the vegetables and fruit. In one embodiment, the reduced calorie diet plan limits caloric intake to 500-800 calories per day. The patient is monitored and the daily HCG dose may be adjusted upward as needed to control hunger and maintain patient comfort.

### DETAILED DESCRIPTION OF THE INVENTION

The present invention is generally directed to a method for weight loss including administering adequate dosing of HCG, and periodically adjusting the dose during the diet regimen to improve results. The method involves administering an initial dose of HCG of greater than 200 IU, providing a reduced calorie diet plan for the patient, monitoring the patients during the diet regimen, and increasing the dose of HCG as needed to control hunger and maintain patient comfort.

The concept behind the dosing regimen is two-fold: (1) administer the starting dose where patients feel comfortable, and (2) increase the dose from that point as needed, since patients seem to develop a “tolerance” to a particular dose after several weeks.

In one embodiment of the invention, the dosing regimen for HCG begins with a starting dose of 200 IU or greater in premenopausal females, and a starting dose of about 250 IU in males or postmenopausal females. This initial dosage regimen is adjusted as necessary while the patient is on the weight loss plan. Dosage adjustments are based primarily upon subjective self-reporting by the patients. Dosage may be increased as necessary to reduce patient discomfort and assist the patient in staying on the low calorie diet plan established for the patient.

In one embodiment of the method, the criteria for adjusting dosage is as follows:
“No Hunger”. This is self-explanatory, and is the ideal response. Sometimes the challenge in these patients is to make sure they are eating enough.
   RESULT: Keep HCG dosage the same as previous week.
“Seldom Hunger”. Very slight hunger between meals. (Note that if a patient gets hungry and it is time to eat, that is normal and does not warrant a dose adjustment.)
   RESULT: Raise the dose 20 IU.
“Frequent Hunger”. Frequent hunger between meals, but tolerable, warrants a raise of the dose 30 IU.
“Always Hungry”. If the patient reports always feeling hungry between meals, and it is not tolerable, raise the dose 50 IU from the previous dose.

In this embodiment, the upper limit of dosing of HCG for weight loss regimens is 330 IU. The invention is not limited in this regard, and the HCG dosage may be increased or adjusted in the methods described herein within proper medical limits in view of potential side effects or other limitations.

In addition to administration of HCG, the weight loss method of the present invention requires adherence to a very low calorie diet comprised of specific foods. The low calorie diet may be established to achieve the desired weight loss while maintaining adequate nutrition for the patient. Typically, the low calorie diet will be in the range of 500-800 calories/day. The HCG/low calorie diet weight loss method may be maintained for as long as the patient desires and tolerates the protocol to achieve a desired weight.

In one embodiment of the invention, a 500 calorie/day diet is established as described below. The diet has the following basic principles: The diet has the following basic principles: (1) eliminate starches and sugars, (2) consumption of minimum required protein for muscle mass, (3) emphasis of low-sugar fruits and vegetables, (4) near zero fats, and (5) a regular LIGHT exercise program. Starch and sugar are power-packed carbohydrates designed for long-term and short-term energy, respectively. When these energy sources are not fully utilized, they are quickly stored in the body as fat. It is essential to avoid pasta, rice, bread, and potatoes, as well as any refined sugar, honey, or sweets. Sugar substitutes (i.e. splenda, stevia) are allowed as part of the diet but in very limited amounts.

Protein and the carbohydrates found in fruit and vegetables, are much more easily used by the body for maintenance and daily supply, and there is rarely anything left over to store as fat. The diet provided stays as close as possible zero fat consumption, including oils. Oil-based substances seem to interfere with the diet, and staying near a fat-free consumption will only benefit the cause. As discussed below, small amounts of cooking oil may be tolerated.

Regular exercise helps metabolism to be strengthened. During any diet, the metabolism has the potential to slow down, which is the body's survival mechanism to conserve itself. A regular exercise regimen, such as power walking for 30 minutes a day, will counteract this metabolism slowing and optimize your weight loss.

If desired, a patient may go off the weight loss protocol for a period of time using the weight maintenance protocol described below, then resume the HCG/low calorie protocol for additional weight loss. Because HCG intercepts hormonal pathways, a rapid withdrawal of the HCG can lead to a temporary reduction in certain hormone levels. Therefore, when a patient goes off the weight loss protocol, whether temporarily or permanently, it may be desirable to gradually reduce the HCG dosage over a period of time. For example, the HCG dosage may be gradually reduced over the course of a week to aid in the patient's return to a natural baseline of hormone levels. Determining the time and HCG dosage reduction rate may be readily determined by one skilled in the art.

In addition to the HCG dosing, there are several features of the weight loss method of the present invention that are different from prior protocols using HCG. For example, the use of oils for cooking is forbidden in the Simeons weight loss protocol. He claimed that a single drop of oil will stall all weight loss for the day. While oils are generally to be avoided due to the density of calories, in one embodiment, small amounts of oil is permitted and should not produce a stall in weight loss.

Prior methods also required reduced or discontinued use of body lotions, cosmetics, shampoo, and other hygiene products. It was thought that body lotions and other cosmetics and hygiene products would cause a stall in weight loss, as they are absorbed into the body and interfere with fat breakdown. In the present method, patients may use all of their usual hygiene products and lose well within the half pound to pound a day range.

Another difference between prior methods and embodiments of the present invention are the protein intake as part of the low calorie diet. Prior diet regimens gave every person, male or female, regardless of weight or height, 3.5 oz of protein twice daily for a total of 7 oz. In one embodiment of the method, a formula is used to establish the protein intake required for the patient individually during the diet phase. The protein is calculated as ideal body weight (in lbs) divided by 2.2 (for weight in kg) then multiplied by 0.8 for females or 1.0 for males. That provides the minimum number of grams of protein per day required for muscle maintenance. If someone is very physically active, or has a BMI greater than 40, the formula used is (weight in kg)×1.0 for females or 1.2 for males. The number of grams of protein per day is divided by 7 to determine the approximate ounces of poultry meat to obtain the grams of protein. For example:

A 220 pound male, whose ideal body weight is 176 pounds, requires: 176/2.2=80 kg, 80/1=80 grams of protein required daily, 80/7=approx 11.5 ounces of chicken daily.

A 220 pound female, whose ideal body weight is 132 pounds, requires: 132/2.2=60 kg, 60/0.8=48 grams of protein required daily, 48/7=approx 7 ounces of chicken daily.

The calorie and protein content in various seafoods and meats is provided in Tables 4 and 5.

Prior methods of weight loss only gave a very limited list of vegetables allowed on the diet, said that “mixing” different vegetables was not allowed, only allowed a single vegetable per sitting. In one embodiment of the present method, a guideline of calories and sugars is used to determine a recommended dietary allowance of vegetables. Using a guideline of sugar and calorie content, a comprehensive list of allowed vegetables has been developed, and vegetables may be mixed, as long as the required calorie and sugar content is not exceeded. In one embodiment, the diet plan allows 50 calories of vegetables containing less than 5 grams of sugar twice per day. Table 2 provides a list of the calorie and sugar content for servings of selected vegetables.

Similarly, prior diet plans using HCG had several limits on fruit that are not part of the present methods. Prior diet plans only allowed: an apple, an orange, a half grapefruit, or a cup of strawberries. In one embodiment, the present method uses similar guidelines as above for vegetables to provide more fruit options. In one embodiment of the method, a fruit snack of about 60 calories per serving may be consumed two or three times per day. The principle is to stay within a low sugar bracket and a low calorie bracket simultaneously. Table 3 provides a list of the calorie and sugar content for servings of selected fruits for the diet plan.

Prior diet methods using HCG stopped the HCG injections during a woman's menses. In the present method, HCG injections are continued during menses because this is the time of the month when appetite increases. When HCG is stopped during menses, hunger can be intense and patients often lose ground from their diet. By continuing HCG injections, patients are able to stay on track with the diet and find their cravings are manageable.

Finally, the length of treatment in prior methods was limited to variations of either 23 days or 46 days. In the present method, a patient may be kept on the protocol for as long as they are responding to treatment, regardless of how many weeks it has been. The longest continuous treatment with the method of the present invention has been more than four months.

While the patient is on the diet protocol, they should be periodically examined. It is important to check blood pressure, weight, and overall appearance of the patient, and ensure that they are tolerating the diet and medication as they were prescribed. Some of the issues that may arise include the following.

Hunger is a common complaint early on. It is important to elucidate whether this is true hunger, or simply “missing” food. The low calorie diet is a drastic reduction in food consumption, which often leads patients to simply miss the act of eating. Additionally, patients may note that they feel “empty”. However, upon further questioning, it may be that they do not feel particularly hungry, just empty since they are not eating a lot. There are those patients that truly feel some degree of hunger between meals, at which time their HCG dose may be adjusted as described above.

Headaches are most commonly due to caffeine withdrawal. When initiating the diet with caffeine drinkers, it is advisable to forewarn them to gradually reduce caffeinated drinks while adding in water so as to not go through a quick withdrawal, which usually produces headaches. Other common reasons for headache are the change in diet which is dramatic for many. If this is the reason, it will typically reverse within a couple of days.

Irritability is most commonly from change in routine, which again will typically reverse in a couple of days. This is a listed side effect of HCG but has been extremely rare to see in practice at the low doses used for weight loss.

Fatigue may occur and can be treated with an IM dose of Vitamin B12, which often makes patients feel markedly better within a day.

Sleep is often improved in length and quality during the protocol. Stamina is sometimes is decreased and sometimes is increased. Most patients report increased energy over the course of the diet.

Noncompliance is extremely rare early in the diet, as this is the time when patients are most motivated and when weight loss is extremely dramatic.

Later in the course of the diet, after several weeks, patients may have a different set of issues that may need troubleshooting.

Most people will reach a point on average around the 3^{rd} or 4^{th} week where a few days may show stalling in weight loss on the scale. The most common reason is water retention. The body requires water in the process of mobilizing fat stores and sometimes the body may hold onto water. It may be that they are not consuming enough water. In one embodiment, the patient is directed to drink at least 100 ounces of water a day, more if they undergo sweating or are in extreme heat. If the body does not receive enough water, it will retain in effort to conserve water. Another common reason for water retention is hormones. Most women will retain water prior to their menses, and often this is the first sign of menses coming on. Therefore when a premenopausal female stalls, it is very important to think about her hormonal state. Lastly, water balance is something that is not entirely perfect and as the body is going through dramatic changes and fat usage, there may be a day or couple of days in which the body is adjusting, and eventually eliminates the excess water. During these phases though, a patient's measurements will still go down significantly, as evidence that they are still using their fat stores. These measurements are very important to alleviate a patient's worries.

Cheating is a more common occurrence as patients spend extended periods of time on the diet. Their thoughts of food they miss lead them to “go off the diet” for a day, or there may be travel involved with poor access to diet-friendly foods. Going off of the recommended diet can lead to stalling through the eating of too much food, or the wrong types of food.

Occasionally patients, despite success, will lose motivation. It is important to recognize this in a patient. At this time they need to be evaluated to see if counseling and encouragement will get them back on track, or if their diet needs to be suspended temporarily. If the patient is not mentally into the protocol anymore, they will not lose anymore, as it takes significant dedication to stay on track, and cheating with the wrong foods or amounts will stall progress.

Prior to starting the protocol, the patient is typically given a physical examination, including blood work and a patient history. Absolute contraindications to HCG treatment include: current cancer of any type (with the exception of skin cancer), suspicious mass being evaluated for a possible cancer, prior sensitivity or allergy to HCG, pregnancy, breastfeeding, unstable medical state (i.e. recent myocardial infarction or cardiac arrest, recent bypass surgery or stenting/angioplasty, recent hospitalization for major medical illness or poorly controlled medical condition), malignant hypertension (>180/120), recent stroke.

Relative contraindications to HCG treatment include: history of a hormone responsive cancer (breast, uterine, ovarian, prostate), poor medical control of any medical ailment, psychiatric instability, newly diagnosed medical conditions or newly prescribed medications, personality disorder (unruly conduct in office), substance abuse (present, not past), active ovarian cyst ready to rupture, body dysmorphic disorder, males with a history of BPH (will require a recent prostate check), females without a recent pap or mammography exam.

### EXAMPLE

The following example describes one embodiment of a diet plan used in conjunction with HCG administration. It should be understood that the following diet plan is exemplary only, and that those skilled in the art may adjust the diet in numerous ways within the scope of the invention described above.

START HCG. The HCG typically takes approximately 2 days to peak in a patient's system. Therefore, for the first two days, the HCG is administered, but the patient does not start the diet. During the first two days of taking HCG, it is advised to “load” or eat in excess. The theory behind the loading is that this stimulates metabolism to facilitate a faster fat loss once dieting is started. Patients also may have a better compliance with the diet when loading occurs beforehand.

START DIET (Day 3 of injections) 500-800 Calories a Day.

BREAKFAST:

| | |
|---|---|
| Coffee, Tea, or Water | 0 cal |

If necessary, 2 egg whites (30 cal) and/or one snack (see below)
Note:
Allowed water substitutes are listed in Table 1.

LUNCH (Protein is calculated for each individual):

| | | |
|---|---|---|
| PROTEIN PORTION: | 100-150 | cal |
| Vegetable serving | 50 | cal |

Note:
Allowed vegetables are summarized in Table 2, and allowed proteins are listed in Tables 4 and 5.

DINNER:

| | | |
|---|---|---|
| PROTEIN PORTION: | 100-150 | cal |
| Vegetable | 50 | cal serving |

SNACKS:

| | | |
|---|---|---|
| “DRY CARB” SNACK- max. 3, anytime of day | 40 | cal/serving |
| Examples: Rice cake, plain Melba toast, | | |
| 3-5 fat-free Saltines | | |
| FRUIT- 2-3 per day, anytime of day | 60 | cal/serving |

Note:
Allowed fruits are listed in Table 3. Allowed condiments and food preparation requirements are summarized in table 6.
(ALL VALUES ARE APPROXIMATE)

Note: There is no allotted breakfast. Upon waking, the patient should weigh themselves and then have a glass of water. Proceed to have usual morning beverage and then wait to see when hunger actually begins. When hunger begins, the patient can have one snack, 2 egg whites, or both. It may be early morning, or mid-morning. More often hunger will first occur several hours after waking.

It is important to get accustomed to calorie counting, especially for the most frequently eaten items. Without calorie counting, it is impossible to know if the daily limit is being exceeded. Completely avoid all fats and oils, cheeses, and nuts. Consume at least 96 oz (12 glasses) of water or water substitutes a day. Avoid caloric beverages.

EXERCISE, 30 minutes of cardio daily are advisable. The heart rate should be elevated to the 120-130 range. Eventually it would be useful to add 30 minutes of weight/resistance training, but is not imperative at the beginning. Instead of weights, toning exercises like Pilates or Yoga work well also. Raising lean muscle mass and lowering fat will increase metabolism and aid the overall process.

TABLE 1
Water Substitutes
100 Ounces of regular water are required daily.
If a substitute is needed, please replace with one of the following:
Water w/lemon
Seltzer
Skinny Water
Pure American Zero Calorie Vitamin Water

Lemonade
Kiwi-Strawberry
Dragon fruit
Acai-Blueberry Pomegranate

Poland Spring Sparkling Water

Original
Mandarin Orange Essence
Lime Essence
Raspberry Lime Essence
Lemon Essence

Other Beverages
(The following DO NOT count toward your water intake for the day)
Coffee/Tea - 1 tbsp of dairy per day; additional coffee black
Limit of one per day
Diet Sodas
Diet Ice Tea
Crystal Light

TABLE 2
Vegetables

| | | | | Calories | Sugars | |
|---|---|---|---|---|---|---|
| Vegetable | Preparation | Serving Size | | (50 cals) | (under 5 g) | |
| Asparagus | Cooked, boiled, | 2⅓ | cup | 47 | 2.7 | g |
| | drained | | | | | |
| Asparagus | Raw | 2 | cups | 54 | 5 | g |
| Broccoli | Cooked, boiled, | 1 | cup | 54 | 2.2 | g |
| | drained | | | | | |
| Broccoli | Raw | 1⅝ | cup | 49 | 2.4 | g |
| Beans, Snap | Cooked, boiled, | 1⅛ | cup | 50 | 2.2 | g |
| | drained | | | | | |
| Beans, Snap | Raw | 1½ | cup | 50 | 2.3 | g |
| Brussel Sprouts | Cooked, boiled, | 1 | cup | 56 | 2.8 | g |
| | drained | | | | | |
| Brussel Sprouts | Raw | 1⅜ | cup | 52 | 2.7 | g |
| Cauliflower | Cooked, boiled, | 3¼ | cup | 47 | 4.2 | g |
| | drained | | | | | |
| Cauliflower | Raw | 2 | cups | 50 | 4.8 | g |
| Cabbage | Cooked, boiled, | 2½ | cups (shredded) | 43 | 5 | g |
| | drained | | | | | |
| Cabbage | Raw | 2½ | cups (shredded) | 42.5 | 5.3 | g |
| Carrots | Cooked, boiled, | 2 | cups (slices) | 54 | 5.4 | g |
| | drained | | | | | |
| Carrots | Raw | ½ | cup chopped | 26 | 5 | g |
| Celery | Raw | | | | | |
| Chives | Scallions | 5 | cups (chopped) | 50 | 0 | g |
| Cucumber | Raw | 3 | cups | 43 | 4.9 | g |
| Cherry tomatoes | Raw | 12 | | 50 | 5. | g |
| Dill Pickle | — | 1 | cup (diced) | 17 | 1.9 | |
| Eggplant | Cooked, boiled, | 1⅜ | cups (1″ cubes) | 45 | 4.4 | g |
| | drained | | | | | |
| Eggplant | Raw | 2⅓ | cup (1″ cubes) | 46 | 4.5 | g |
| Lettuce, Iceberg | Raw (fresh) | 5 | cups (shredded) | 40 | 5.4 | g |
| Lettuce, Romaine | Spring Mix | 6 | cups | 45 | 2.9 | g |
| Mushrooms | Raw | 3 | cups (slices) | 47 | 3.5 | g |
| Mushrooms | Cooked, boiled, | 1 | cup (pieces) | 44 | 3 | g |
| | drained | | | | | |
| Onion, Red | Raw | ½ | cup | 34 | 3.5 | g |
| Onion, White | Raw | ½ | cup | 34 | 3.5 | g |
| Pepper, Red | Raw | ½ | cup (chopped) | 23 | 3.1 | g |
| Pepper, Red | Cooked, boiled, | ½ | cup (chopped) | 19 | 3 | g |
| | drained | | | | | |
| Pepper, Green | Raw | 1 | cup (chopped) | 30 | 3.6 | g |
| Pepper, Green | Cooked, boiled, | 1 | cup (chopped) | 38 | 4.3 | g |
| | drained | | | | | |
| Radish | Raw | 10 | each | 17 | 3.3 | g |
| Radish | Cooked, boiled, | 2 | cups (sliced) | 50 | 5.4 | g |
| | drained | | | | | |
| Spinach | Raw | 7 | cups | 49 | 0.9 | g |
| Spinach | Cooked, boiled, | 1⅛ | cup | 47 | 0.9 | g |
| | drained | | | | | |
| Squash(summer, | Cooked, boiled, | 1½ | cup slices | 43 | 4.6 | g |
| zucchini, skin) | drained | | | | | |
| Squash (summer, | Raw | 2 | cups (slices) | 37 | 5 | g |
| zucchini, skin) | | | | | | |
| Tomato | Raw | 1 | cup (chopped) | 32 | 5 | g |

TABLE 3
Fruit

| | | | Calories | Sugars | |
|---|---|---|---|---|---|
| Fruit | Serving size | | (80 cals) | (under 12 g) | |
| Apple-Delicious | 1 | cup (sliced) | 58 | 11.4 | g |
| Apples* | | | | | |
| Blackberries | 1 | cup | 62 | 7 | g |
| Blueberries | ½ | cup | 42 | 7.2 | g |
| Cherries | ½ | cup | 45 | 9 | g |
| Clementine | 1 | | 40 | 6.5 | g |
| Cranberries | 1 | cup | 60 | 10 | g |
| Grapefruit | ½ | of grapefruit | 52 | 8.5 | g |
| Grape, Red | ½ | cup | 60 | 12 | g |
| Grape, Green | ⅜ | cup | 45.6 | 10 | g |
| Kiwi | 1 | medium | 50 | 8 | g |
| Lemon | 1 | medium | 15 | 1 | g |
| Lime | 2″ | cut | 20 | 1.1 | g |
| Mango | ½ | cup | 53.5 | 12.2 | g |
| Melon | ½ | cup (cubed) | 30 | 7 | g |
| Nectarine | 1 | cup (slices) | 61 | 11 | g |
| Orange | 1 | medium | 62 | 12.2 | g |
| Papaya | 1 | cup | 55 | 9 | g |
| Peach | 1 | medium | 38 | 8.2 | g |
| Pear | ½ | cup | 48 | 8.2 | g |
| Pineapple (fresh) | ½ | cup | 39 | 7.7 | g |
| Plum | 1 | medium | 30 | 6.5 | g |
| Strawberry | 1 | cup (sliced) | 53 | 7 | g |
| Watermelon | 1 | cup (diced) | 46 | 9 | g |

TABLE 4
Seafood

| | Serving | | Fat | | | | |
|---|---|---|---|---|---|---|---|
| Fish | Size | | (under 2 g) | | Calories | Cholesterol | |
| Blue Crab | 3 | oz. | 1 | g | 100 | 95 | mg |
| Clams | 1 | cup | 2.2 | g | 168 | 77 | mg |
| Cod | 3 | oz. | 1 | g | 90 | 50 | mg |
| Flounder/sole | 3 | oz. | 1.5 | g | 100 | 55 | mg |
| Haddock | 3 | oz. | 1 | g | 100 | 70 | mg |
| Halibut | 3 | oz. | 2 | g | 120 | 40 | mg |
| Lobster | 3 | oz. | 0.5 | g | 80 | 60 | mg |
| Ocean Perch | 3 | oz. | 2 | g | 110 | 45 | mg |
| Pollack | 3 | oz. | 1 | g | 90 | 80 | mg |
| Rockfish | 3 | oz. | 2 | g | 110 | 40 | mg |
| Scallops | 3 | oz. | 0.6 | g | 75 | 28 | mg |
| Shrimp | 3 | oz. | 1.5 | g | 100 | 170 | mg |
| Tilapia | 3 | oz. | 2.5 | g | 110 | 75 | mg |
| Tuna | 3 | oz. | 1.5 | g | 130 | 50 | mg |
| (fresh or canned | | | | | | | |
| in water) | | | | | | | |

TABLE 5
VERY LEAN MEAT
Chicken and 97% Lean Beef
*BONELESS, SKINLESS, CHICKEN BREAST *

| | | | | |
|---|---|---|---|---|
| | Serving | | | |
| Name | Size | Fat | Calories | Protein |

Beef and Pork

| | | | | | | |
|---|---|---|---|---|---|---|
| Lean Ground Beef | 3 | oz. | 3.4 | g | 105 | 18 |
| London Broil Roast Beef 97% | 3 | oz. | 2.3 | g | 90 | 18 |
| Lean | | | | | | |
| Venison (Deer) | 3 | oz. | 1.8 | g | 138 | 29.4 |
| Pork, Fresh, Loin, Tenderloin | 3 | oz. | 3 | g | 122 | 22.2 |

Chicken and Turkey

| | | | | | | |
|---|---|---|---|---|---|---|
| Boneless, Skinless, Chicken | 3.5 | oz | 1.3 | g | 105 | 23 |
| Breast (Organic Valley) | | | | | | |
| Boneless, Skinless, Chicken | 3.5 | oz | 1.1 | g | 95 | 21 |
| Breast (Perdue) | | | | | | |
| Gourmet Lite Turkey Breast | 3 | oz. | 1.5 | g | 75 | 15 |
| (Dietz & Watson) | | | | | | |
| Extra Lean Ground Turkey | 3.5 | oz. | 1.3 | g | 105 | 23 |
| Breast (Jennie-O) | | | | | | |

TABLE 6
Condiments & Food Preparation

CONDIMENTS
Hot Peppers
Hot Sauce
Low-Carb Ketchup
Mustard, Dijon
Relish
Salsa (plain- hot or mild)
Soy Sauce, Low Sodium
Walden Farms Dressing
Sugar-Free Gum
Vinegar (any variety: apple cider,
balsamic, red wine, etc.)
Lemon Juice/Lime Juice
Spices and seasonings
FOOD PREPARATION
Boil, Bake, Grill, Poach, Steam, Broil
No cooking sprays or oils of any kind

I claim:

1. A method for weight loss in a person comprising the steps of:
a) administering HCG to the person at a daily dose of at least 200 IU; and
b) providing a reduced calorie daily diet plan to the person.

2. The method of claim 1, wherein the method further comprises the steps of:
c) monitoring the person on a regular periodic schedule; and
d) increasing the daily dose of HCG periodically to control discomfort caused by the diet plan.

3. The method of claim 2, wherein the reduced calorie daily diet plan is between 500 to 800 calories per day.

4. The method of claim 4, wherein the reduced calorie daily diet plan is 500 calories per day.

5. The method of claim 1, wherein the daily dose of HCG is about 250 IU.

6. The method of claim 3, wherein the daily dose of HCG is increased by 20 to 50 IU.

7. The method of claim 3, wherein the daily dose of HCG is increased to a maximum of 330 IU.

8. The method of claim 3, wherein the amount of protein included in the diet plan is determined based upon the ideal body weight of the patient.

9. The method of claim 3, wherein the diet plan includes two servings of vegetables per day with each serving having about 50 calories.

10. The method of claim 3, wherein the diet plan includes two to three servings of fruit per day with each serving having about 60 calories.
